(19) United States
(12) Patent Application Publication
(10) Pub. No.: US 2005/0175702 A1
(43) Pub. Date: Aug. 11, 2005

(54) Thermosensitive polymer carriers having a modifiable physical structure for biochemical analysis, diagnosis and therapy

(76) Inventor: Detlef P Muller-Schulte, Aachen (DE)

Correspondence Address: D Peter Hochberg;D Peter Hochberg Co, 1940 East 6th Street, 6th Floor, Cleveland, OH, 44114, US

(21) Appl. No.: 10/516,629

(22) PCT Filed: May 28, 2003
(86) PCT No.: PCT/EP03/05614

(30) Foreign Application Priority Data
Jun. 1, 2002 (DE) 102 24 352.2

(51) Int. Cl. A61K 48/00; A61K 38/43; A61K 39/395; A61K 9/14; A61K 33/24

(52) U.S. Cl. 424/486; 424/130.1; 514/44; 424/94.1; 424/650

(57) ABSTRACT

The invention relates to thermosensitive polymers which contain magnetic and/or metallic colloids and whose physical structure can be altered through magnetic induction or through the supply of energy. The invention also relates to processes for the production of such thermosensitive polymers, and the use of such polymers for diagnostic and therapeutic purposes.

### CROSS REFERENCE TO RELATED APPLICATIONS

This application is a National Stage application of International Application No. PCT/EP03/05614, filed on May 28, 2003, which claims priority of German application number 102 24 352.2, filed on Jun. 1, 2002.

### BACKGROUND OF THE INVENTION

1. Field of the Invention

The invention relates to thermosensitive polymers that can be heated by magnetic induction on account of encapsulated magnetic and/or metallic colloids and thus experience a change in their physical structure or form. The change in form that accompanies the heating is used amongst others to produce controllable drug depots, contrast-intensifying media for NMR diagnostics, manipulable micro-tools, as a means to block blood vessels and as controllable porogens during membrane production.

The invention relates to polymer carriers of various geometries and particle sizes into which a magnetizable and/or metallic substance or a magnetic and/or metallic core polymer containing a colloid have been polymerized that can be selectively heated through the introduction of heat or in a high-frequency, magnetic alternating field resulting in a change in the physical structure and/or shape of the polymer carrier that enables an in vivo application of the said polymer carriers. The invention also relates to the production and use of the polymer carriers.

2. Description of the Prior Art

Magnetic polymer particles that can be heated by induction are described in various publications and patents. For example, DE-OS 3502998, DE-OS 4201461, DE-OS 4412651 and DE-OS 19800294 describe magnetic polymer particles that can be inductively heated for tumor therapy, for AIDS therapy and for molecular-biology applications.

Jordan et al., J. Magnet. Mag. Mat., Vol. 225, 118, 2001, and Int. J. of Hyperthermia, Vol 9, 51, 1993 use variously coated magnetic colloids that can be heated inductively for the hyperthermic treatment of tumors. In a similar fashion, Mitsumori et al., Int. J. of Hyperthermia, Vol. 10, 785, 1994, and Masuko et al., Biol. Pharm.Bull., Vol. 18, 1802, 1995, use magnetic particles that can be heated by induction and magnetic liposomes to overheat (hyperthermia) tumor cells.

The U.S. Pat. Nos. 4,735,796 and 4,662,359 describe magnetic particles that are also used for hyperthermia in the context of tumor therapy.

A common feature of the media and processes cited here is that magnetic induction is used solely to heat the particles so as to destroy cells or biological organisms by overheating. A change in the physical structure or form of the polymer carrier with the aid of induction cannot be realized with the known carriers.

Magnetic micro and nano-particles preferably for analytical, diagnostic or medical purposes are generally known from the patents PCT/WO 97/04862, PCT/WO 89/11154, PCT/WO 92/22201, PCT/WO 90/07380, PCT/WO 99/62079 and the U.S. Pat. Nos. 6,020,210, 5,141,740, 4,827,945, 4,647,447, 3,917,538, 4,628,037, 4,827,945, 4,861,705, 4,169,804, 4,115,534, 4,345,588, 4,070,246, 3,970,518, 4,230,685, 4,654,267, 4,452,773, 4,369,226, 4,357,259, 4,861,705, 4,247,406, 4,267,234, 3,652,761, 4,675,173, enclosed herewith as a reference. Dextran, agarose, dextrin, albumin, silica gel, polystyrene, gelatin, polyglutaraldehyde, agarose-polyaldehyde composites, liposomes, polyethyleneimine, polyvinyl alcohol, polyacrolein, proteins and polyoxyethylenes are used as a polymer matrix in the aforementioned processes and products, these being able to bond analytes via coupled bioligands and/or receptors according to the principle of affinity in the form of antigens, antibodies, proteins, cells, DNA fragments, viruses or bacteria in the context of biochemical-medical analytics and diagnostics.

An overview of further magnetic particles coated with different polymers and their application in the field of biomedicine are described in “Scientific and Clinical Applications of Magnetic Carriers”, Hafeli et al., published by Plenum Press, New York, 1997.

A common feature of all of the aforementioned products is that they derive their function exclusively from the complementary interaction of a bioligand or receptor bonded to the matrix with the substance to be analyzed. Their fields of use are thus restricted to the known fields of the separation and analysis of biomolecules or the marking of certain cells using the principle of affinity.

The magnetic polymer carriers cited here as references also differ from the media in accordance with the invention in that on account of their chemical structure they are not thermosensitive, i.e. they are not able to change their physical structure or geometric form on the basis of an external thermal stimulus. This property however is the basic condition for using polymer carriers as manipulable or controllable micro or nano carriers and/or tools.

The most commonly used polymer with thermosensitive properties is poly-N-isopropylacrylamide, a gel-like polymer that experiences a significant shrinkage at temperatures above 27° C. This shrinkage is reversible, i.e. if cooled to below 30° C. the polymer practically resumes its original form. This special property of poly-N-isopropylacrylamide and the interesting applications that can be derived from this, for example as a drug depot, biosensor, cell culture substrate, cell encapsulation matrix, actuator or valve have been known for a long time and are reflected in a number of publications and patents.

The polymerization and swelling properties of N-isopropylacrylamide or copolymers with, for example, acrylic acid, methylacrylic acid, polyethylene oxide or chitosan as well as graft copolymerization with silicone rubber or polyvinyl alcohol are described by Park and Hoffman, J. Biomed. Mat. Res., Vol. 24, 21, 1990, Zhang et al., Langmuir, Vol. 18, 2013, 2002, Lee and Chen, J. Appl. Polymer Sci., Vol. 82, 2487, 2001, Zhu and Napper, Langmuir, Vol. 16, 8543, 2000, Li et al., Radiat. Phys. Chem., Vol. 55, 173, 1999, Zhang and Zhuo, Eur. Polym J., Vol. 36, 2301, 2000, Serizawa et al., Macromolecules, Vol. 35, 10, 2002, Kanazawa et al., Anal. Sci., Vol. 18, 45, 2002, Asano et al., Polym. Gels & Network, Vol. 3, 281, 1995, Sayil and Okay, Polym. Bull., Vol. 45, 175, 2000, Xue et al., Polymer, Vol. 42, 3665, 2001, Maolin et al., Radiat. Phys. Chem., Vol. 57, 481, 2000, as well as Ebara et al., J. Appl. Polymer Sci., Vol. 39, 335, 2001. Corresponding nano-particles or microparticles of poly-N-isopropylacrylamide as a base polymer are described by Gan and Lyon, J. Am. Chem. Soc., Vol. 123, 7517, 2001, Wang et al., J. Am. Chem. Soc., Vol. 123, 11284, 2001, Gilanyi et al., Langmuir, Vol. 17, 4764, 2001, West and Halas, Curr. Opinion. Biotechn., Vol. 11, 215, 2000, Matsuoka et al., Polym. Gels & Networks, Vol. 6, 319, 1998 and Jones and Lyon, Macromolecules, Vol. 33, 8301, 2000.

The subject of the work by Kondo and Fukuda, J. Ferment. Bioeng., Vol. 84, 337, 1997, are N-isopropylacrylamide copolymers containing magnetic nano-particles. However, the process described therein provides neither clear magnetic particle encapsulations nor spherical particles. Kondo et al., Appl. Microbiol. Biotechn., Vol. 41, 99, 1994, describe magnetic polystyrene polymers that are encapsulated in a time-consuming two-stage polymerization with poly-N-isopropylacrylamide-methyl acrylic acid copolymers. The products are only suitable to separate antibodies. An active agent application in connection with an inductively controlled release of the same, such as is the subject of this present invention, cannot be realized with either product.

The U.S. Pat. Nos. 4,832,466, 6,165,389, 6,187,599, 5,898,004, 5,854,078, 6,094,273, 6,097,530, 5,711,884 and 6,014,246 disclose thermosensitive optical systems in the form of filters or switches, etc, using poly-N-isopropylacrylamide nano-particles.

The U.S. patent applications 20020032246, 20020031841, 20010026946 describe amongst others thermosensitive hydrogels, etc., on the basis of N-isopropylacrylamide for the separation of macromolecules, for the colorimetric detection of analytes or as sensors to determine chemical compounds.

Thermo- and pH-sensitive polymer gels from, amongst others, N-isopropylacrylamide, hydroxyalkylcellulose, polyethylene oxide, polyethylene glycol, polyvinyl alcohol, dextran, alkyl cellulose, block polymers of polyoxyethylene, polyoxypropylene, polyacrylic acid, ethylene diamine e.g. as carriers for biologically active substances are mentioned in the U.S. Pat. Nos. 5,674,521, 5,441,732, 5,252,318, 5,599,534, 5,618,800 and 5,840,338.

Temperature and pH-sensitive polymers of interpenetrating polymer networks, consisting amongst others of acrylates, acrylamides, urethanes or methacrylates and block copolymers of polyoxyethylene or polyoxypropylene, are the subject matter of the U.S. Pat. No. 5,939,485.

U.S. Pat. No. 5,998,588 describes light, temperature and pH-sensitive interactive stimulus-response molecule conjugates of poly-N-isopropylacrylamide for assays or separations. pH, light and temperature-sensitive lipid-coated microparticles as well as microparticles and liposomes of N-substituted polyacrylamides as a drug depot are also disclosed in the U.S. Pat. Nos. 5,753,261, 5,226,902 and 5,053,228.

Porous carrier media of rayon, paper, polyacrylamide and agarose beads, etc., as a solid phase carrier to detect analytes are described in U.S. Pat. No. 5,013,669.

Enzymes immobilized on acrylate carriers with reversible solubilities are the subject matter of U.S. Pat. No. 4,783,409.

A thermally induced phase-separation-immunoassay using poly-N-isopropylacrylamide copolymers to detect drugs, hormones, vitamins, proteins, metabolites, cells, viruses, microorganisms and antibodies is disclosed in U.S. Pat. No. 4,780,409.

The synthesis and application of antibody-polymer conjugates based on N-hydroxysuccinimide acrylates in the context of the immunoassay as well as for analytical purposes are published in the U.S. Pat. Nos. 4,752,638 and 4,609,707.

Temperature-sensitive poly-N-isopropylacrylamide or poly-N-isopropylacrylamide copolymers containing receptors, antibodies, proteins, drugs or nucleic acid that are able amongst others to release active agents are the subject matter of U.S. Pat. No. 4,912,032.

Alginate beads as an oral drug depot are described in U.S. Pat. No. 5,451,411.

Biodegradable shape-memory-polymers consisting of hard and soft polymer segments and whose original shape can be restored by heating to above the glass transition temperature are the subject mater of U.S. Pat. No. 6,160,084.

All of the aforementioned media listed here as a reference have one thing in common, namely that wherever these are non-magnetic polymer carriers, a change in the physical structure or form can only be triggered through heat that is applied directly from the outside, and that wherever they are magnetic carriers, their structure cannot be changed in any way, neither through an external stimulus nor through externally applied energy. Furthermore, the “stimulus-response” carriers known from the state-of-the-art are either irregular nano-particles or larger volume mass polymers that are unsuitable as carriers for active agents (drugs), as a contrast medium in NMR diagnostics (magnetic resonance tomograph), as media for molecular separation or as controllable micro-tools for in vivo applications.

### SUMMARY OF THE INVENTION

The object of the present invention is to provide polymer matrices and/or polymer carriers in a nano or microparticle form as well as other geometries that can be selectively stimulated by an energy supply, e.g. in the form of magnetic induction, to induce a parallel, defined change in the physical structure of the polymer matrix on the basis of the resulting increase in temperature.

By definition, a “change in the physical structure” is hereby understood as meaning a change in the geometric shape, volume or particle size of the polymer carrier. The change in volume may be manifested for example in a shrinkage or swelling process with a parallel change in the pore size or in a change of the external form (geometry) of the polymers. Changes in the physical structure can also mean that the polymer returns to its original form that has been temporarily changed through a heating and cooling process (freezing process) (“shape-memory-polymer”).

Since the phase transition temperature (also: “critical solution temperature”) of these polymers is in the range of 27-38° C., i.e. in the body temperature range (37° C.), these carriers could not as yet be used in vivo since the shrinkage process has already occurred at this temperature and/or the carrier cannot be heated up any more. In order to make the carriers based on poly-N-isopropylacrylamide and N-substituted acrylamides useful for an in vivo application as carriers for active agents of a therapeutic, analytical and diagnostic type, a further object of the invention is to encapsulate active agents in the polymer carrier and after corresponding in vivo administration to apply these selectively and controllably with the aid of magnetic induction.

The combination of heating induced by the magnetic field with a parallel change in the physical structure and/or carrier geometry should create a range of properties that go far beyond former polymer carrier systems.

The object of the invention is solved by heating certain polymers through magnetic induction, i.e. through an externally applied, high-frequency magnetic alternating field, by encapsulating magnetic and/or metallic substances in the polymer matrix that are able to absorb energy from the magnetic field and can heat up the polymer carrier accordingly.

The object is also solved in accordance with the invention by synthesizing special polymers and copolymers on the basis of poly-N-isopropylacrylamide and N-substituted acrylamides that react to the thermal stimulus by changing their physical structure.

### DETAILED DESCRIPTION OF THE INVENTION

The initial product to produce the thermosensitive polymer carriers are magnetic colloids in the form of ferromagnetic, ferrimagnetic or superparamagnetic nano or microparticles that display a high magnetization and can be inductively heated in a magnetic alternating field. The preferred substance for this purpose is magnetite (Fe₃O₄) or γ-Fe₂O₃. The production of such compounds is known from the general state-of-the-art: Shinkai et al., Biocatalysis, Vol. 5, 61, 1991, Kondo et al., Appl. Microbiol. Biotechnol., Vol. 41, 99, 1994, Khalafalla and Reimers, IEEE Trans. Magn., Vol. 16, 178, 1980, Lee et al., IEEE Trans. Magn., Vol. 28, 3180, 1992, Buske et al., Colloids & Surfaces, Vol. 12, 195, 1984.

Colloidal dispersions of magnetite or γ-Fe₂O₃ without using any stabilisers have been published by Kang et al., Chem. Mater., Vol. 8, 2209, 1996.

Similar magnetic colloids that consist primarily of magnetite (Fe₃O₄), iron oxide (Fe₂O₃) or iron oxyhydroxide (FeOOH) and have a particle size of 5-100 nm and are used amongst others as contrast media in NMR diagnostics, as information storage media, sealants or suppressants or for cell marking, can be found in the following patents, enclosed here as a reference: U.S. Pat. Nos. 5,492,814, 5,221,322, 4,647,447, 4,827,945, 4,329,241, 3,215,572, 3,917,538, DE-OS 350 8000, DE-OS 39 33 210, DE-OS 39 33 210, EP 0 275 285, PCT/IL99/00275, EP 0 586 052.

Other substances that have the aforementioned properties and are thus suitable for encapsulation in a polymer matrix include for example ferrites with the general formula MOFe₂O₃, whereby M is a bivalent metal ion or a mixture of two bivalent metal ions or metallic nickel or cobalt.

Iron (III) and iron (II) saline solutions with varying molar ratios (2:1, 0.5:1 to 4:1) form the basis of producing magnetite or γ-Fe₂O₃, these then being converted into corresponding colloidal magnetic dispersions (“magnetic colloids”) by adding bases or applying heat. In order to prevent an agglomeration of the fine magnetic particles due to the van-der-Waals-forces, surface active agents can be added that are generally known under the names “tensides”, “emulsifiers” or “stabilisers” that practically prevent a precipitation of the colloid in an aqueous dispersion. Such stabilizing colloidal dispersions are also known under the name “ferrofluids” (cf. Kaiser and Miskolczy, J. Appl. Phys., 413, 1064, 1970). They are also available commercially (Ferrofluidics Corp., USA; Advanced Magnetics, USA; Taibo Co, Japan; Liquids Research Ltd., Wales; Schering AG, Gemany).

The stabilisers used are either cationic, anionic or non-ionic. Suitable compounds for these include, e.g.: alkyl aryl polyether sulfates, lauryl sulphonate, alkyl aryl polyether sulphonates, phosphate ester, alcohol ether sulfates, citrates, oleic acid, alkyl naphthalene sulphonates, polystyrene sulphonic acid, polyacrylic acid or petroleum sulphonates as anionic substances, dodecyl trimethylammonium chloride as a cationic tenside and nonyl phenoxypolyglycidole, polyvinyl alcohol, kerosene, alkyl aryl oxypolyethoxy ethanol, nonyl phenol or polyethylene glycols as non-ionic substances. The particle sizes of the magnetic colloids produced by the aforementioned preparation methods depend, as is generally known (see cited references), on various test parameters such as the iron salt ratio, base concentrations, pH value and temperature.

The magnetic colloids suitable for the media in accordance with the invention all have a particle size of 5-1000 nm, preferably one of 10-500 nm. This guarantees that the magnetic colloids are present in a finely dispersed form during subsequent encapsulation in the polymer matrix. Through a selective, metered addition of corresponding amounts of the appropriate colloid, the magnetic properties and analogously the heat-up properties of the polymer carrier can be specifically controlled.

The concentrations of magnetic colloids in the monomer formulation are normally 10 to 30% by volume, whereby the solid content of the magnetic substance relative to the monomer phase is generally 5 to 40% by weight, preferably 10 to 30%.

Apart from magnetic colloids, metallic colloids can also be encapsulated in the polymer matrix as an alternative. All metallic substances in a colloidal or finely dispersed form that can be inductively heated in a high-frequency, alternating field are in principle suitable. Since physiological applications of the media in accordance with the invention represent an essential aspect, those metal colloids that can be inductively heated which are physiologically harmless and/or chemically-physically inert are preferably used. These include the metals in groups 8 to 11 (IUPAC definition 1986), whereby gold, silver, palladium and platinum colloids or corresponding powders are preferably used on account of their biocompatibility.

The metal colloids used for the media in accordance with the invention normally have a particle size of between 5 and 300 nm. The production of such colloids, that have long been used to determine proteins and nucleic acids on account of their special absorption properties in the visible range in bioanalytics, above all the gold colloids, is sufficiently known from the state of the art: Ackerman et al., J. Histochem. Cytochem., Vol. 31, 433, 1983, Geoghagen et al., J. Histochem. Cytochem., Vol. 24, 1187, 1977, Wang et al., Histochem., Vol. 83, 109, 1985, Birell et al., J. Histochem. Cytochem., Vol. 35, 843, 1987, Kohler et al., Sensors & Actuators B, Vol. 76, 166, 2001, Moeremans et al., Anal. Biochem., Vol. 145, 315, 1985, Englebienne, Analyst, Vol. 123, 1599, 1998, and U.S. Pat. No. 6,361,944. As any expert in this field knows, they are all produced through the reduction of the corresponding metallic salts or by the metal spraying method. A large number of metal colloids or powders are also available commercially (Sigma, Aldrich, Fluka).

Both the metal colloids and corresponding powders can be used for the media and processes in accordance with the invention; these are admixed to the monomer formulation in the desired concentration before polymerization. The metal shares in the polymers and/or particles are normally between 5 and 40% by weight.

After adding the colloids, it is often advantageous to briefly expose the colloid-monomer mixture to ultrasonic waves using an ultrasonic finger or ultrasonic bath to ensure a fine dispersion of the colloid. The homogeneous distribution of the colloid enables a correspondingly better dissipation of heat in the polymer matrix, which in turn guarantees a continuous release of the encapsulated active agent.

N-isopropylacrylamide and/or N-substituted acrylamides such as N-cyclopropyl acrylamide, N-cyclopropyl methacrylamide, N,N-diethyl acrylamide, N-n-propyl methacrylamide, N-isopropyl methacrylamide, N,N′-ethyl methyl acrylamide, N-ethyl acrylamide, propyl methacrylamide as well as N-acryloyl pyrrolidone or N-acryloyl piperidine are used as thermosensitive monomers for the polymer matrix and for the nano or microparticle carrier. As is known (cf. references), poly-N-isopropylacrylamide has a phase transition temperature between 27 and 38/40° C. on account of its special chemical structure, and this induces a clear shrinking process in the gel above this temperature.

In order to produce the thermosensitive polymers that are normally used as 5-30% solutions, two basic methods are used depending on the form and intended use of the polymer carrier:
radical polymerization in solution; and
radical polymerization in a dispersed state.

The latter include the familiar methods such as pearl, suspension, emulsion, spray and precipitation polymerization to produce finely dispersed polymer particles. Polymerization in a dispersion or suspension has proven particularly advantageous to produce the media in accordance with the invention, where the monomer mixture is suspended by stirring together with the corresponding colloid in an organic phase that cannot be mixed with water and hereby radically polymerized (“inverse suspension polymerization”).

Examples for such organic phases used in suspension polymerization are generally known: Johansson and Mosbach, Biochem. Biophys. Acta, Vol. 370, 339, 1974. Aromatic hydrocarbons such as toluene or benzene, chlorinated hydrocarbons, aliphatic hydrocarbons or mineral and vegetable oils are primarily used here.

Surprisingly, hydrocarbons with a polar solubility parameter of 5-10 (cal/cm³)^{1/2} have proven particularly suitable for media and processes in accordance with the invention, whereby the solubility parameters quoted by K. L. Hoy (“Tables of Solubility Parameters”, Union Carbide Corporation, South Charleston, 1969) have been taken as a basis for the present invention. Examples in the sense of the invention are: 1,2-dichloropropane, 1,1,2-trichloroethane, trichloroethylene, bromotrichlomethane, tetrachloromethane, 1,1,1,2-tetrachloroethane, chloroform, 2,3-dichloropropanol, 1,2,3-trichloropropane.

Apart from the use of the corresponding organic solvents, the quality of the polymer particles with respect to dispersability is expedited by the addition of certain surfactant substances. Examples of these that do not restrict the invention include: derivatives of polyoxyethylene adducts, alkyl sulphosuccinates, polyoxyethylene sorbitol ester, polyethylene propylene oxide block copolymers, alkyl phenoxypolyethoxy ethanols, fatty alcohol glycol ether phosphoric ester, sorbitan fatty acid ester, sucrose stearate palmitate, fatty alcohol polyethylene glycol ether, polyglycerol ester, polyoxyethylene alcohols, polyoxyethylene sorbitan fatty acid ester and polyoxyethylene acids. In order to reduce the size of the polymer drops produced by the suspension process to <1 μm, 0.3-15% by weight, preferably 0.5-5% by weight of one or more surfactants are normally added to the dispersion phase.

These particle sizes are suitable above all for a biomedical in vivo application. Particles with a size of 20-200 nm are preferably used as contrast media in DNA diagnostics and as porogens to produce adjustable pore widths in membranes, those between 100-500 nm particularly as drug depots for the selective application of active agents, e.g. in the form of therapeutic, diagnostic or prophylactic agents. These particle sizes lastingly support the ability to penetrate tissue for in vivo applications. A similar situation applies if the poly-N-isopropylacrylamide particle is used as a medium to set defined pore widths in membranes. By intercalating poly-N-isopropylacrylamide nano-particles in a random plastic matrix, pores can be created whose size can be reduced and enlarged between 10% and 80% through inductive heating and subsequent cooling.

The dispersion process is normally carried out with a conventional KPG stirrer or a dispersing machine. Conventional propeller mixers with stirring speeds of between 600 and 1500 rpm are adequate for particle sizes of between 10-500 μm. Particle sizes <10 μm are normally realized by stirring speeds of >1500 rpm. On the other hand, only dispersing machines with mixing speeds of >2000 rpm are needed for particle sizes of <1 μm. All stirrers that work according to the rotor-stator principle are used for this purpose. At these high mixing speeds the experiments are preferably carried out in an argon or nitrogen atmosphere or in a vacuum to largely rule out the introduction of air that could permanently affect the dispersion quality.

For applications in the biomedical field, in particular, it could be demonstrated that homopolymers of poly-N-isopropylacrylamide alone cannot be used for an in vivo application. This is related to the phase transition temperature of the poly-N-isopropylacrylamide, which is generally between 27 and 38° C. for manufacturing reasons. Since these temperatures are already below the normal body temperature this means that the intended and application-relevant changes in the physical structure in the polymer gel have already occurred. To allow these specific properties nevertheless to be used for the in vivo application, it could surprisingly be shown that the phase transition temperature can be raised through a copolymerisation of the N-isopropylacrylamide with co-monomers containing carboxyl groups so that the function of the media in accordance with the invention can be fully exploited in conjunction with the inductive heating.

Thus, nano and microparticle acrylic acid and methacrylic acid copolymers whose co-monomer content is between 0.02 and 3% by mol display a maximum shrinkage above 40° C. As a result of the incorporation of the partially charged carboxyl groups there is a basic swelling of the polymer gels so that the hydrophobic interactive forces that normally cause the gel to shrink are greatly reduced with a rising temperature. Microparticle gels with a mean particle size of 3.4 μm and an acrylic acid content of 1% by mol display a reduction of the hydrodynamic particle diameter of 18% compared to the value at room temperature (20° C.) after 4 minutes at 38° C. under neutral pH-conditions, and on the other hand the same carrier displays a shrinkage rate of >40% at >45° C. with otherwise identical conditions.

Apart from the degree of shrinkage, higher temperatures also accelerate the shrinkage kinetics. Thus, the shrinkage process at 45° C. is normally faster than that at 35° C. by the factor 1.5 to 3.

The phenomenon of a swelling of the polymer gels due to co-monomers containing carboxyl groups can also be used to optimize the pore sizes and pore structures of the gels to the respective encapsulation tasks. Higher-molecular biomolecules such as IgM antibodies or the enzyme galactosidase, that has a molecular mass of >500 kDa, are generally unable to diffuse out of a N-isopropylacrylamide homopolymer in a reasonable length of time (a few minutes). The pore channels in this case are too small. Thanks to the described copolymerisation with carboxyl group co-monomers, the pores can however be dilated to enable a diffusion for such biomolecules too. Co-monomer contents of 0.01 to 2% by mol are normally sufficient to induce the necessary structural and property modalities.

Apart from copolymerisation, as the parameter that supports the dynamics of shrinkage, certain substances that are added to the monomer mixture before polymerization may surprisingly contribute to a pore dilation and acceleration in the shrinkage process. Substances of this type that normally occur in a concentration of between 2 and 30% by weight, preferably between 2 and 20% by weight, are for example nano-scale silica particles that can be manufactured, e.g., according to a method from Stober et al., J. Colloid Interface Sci., Vol. 26, 62, 1968, as well as polyethylene glycols or polyethylene oxides, in each case with a molecular mass between 200 and 5000, moreover polysaccharides or modified polysaccharides with a molecular mass between 500 and 10,000. Thus, 5-15% by volume (relative to the monomer solution) of poly-N-isopropylacrylamide particles (mean particle size 18 μm) that contain polyethylene glycol (molecular mass 400) generally lose 5 to 20% of water within 3 minutes when heated to >45° C. whereas the same particles with a polyethylene glycol content of >30% lose between 50 and 80% of their water content within 3 minutes under otherwise identical conditions. This increased loss of water with an increasing polyethylene glycol content is also accompanied by an analogous increase in the dynamics of shrinkage that has a direct effect on the release kinetics of the active agents encapsulated in the polymer carrier. Thus, the active agent release can generally be accelerated by a factor of 1.5 to 5 by adding such porogens.

A further method to produce nano and microparticle polymer carriers is to graft N-isopropylacrylamide onto a previously synthesized, spherical, magnetic polymer core or to surround and encapsulate this with poly-N-isopropylacrylamide during the polymerization process. This means that apart from the suspension polymerization described above, other polymerization variants such as precipitation polymerization and emulsion polymerization can be used to produce the media in accordance with the invention. This also opens up the possibility of obtaining ideal-spherical and monodisperse carriers such as are produced, in particular, by emulsion polymerization. New product properties can also be realized with the aid of this process and product combination that significantly extend the range of applications for the new carriers. For example, rigid core polymers such as polystyrene, polystyrene copolymers, polymethyl methacrylate, polyglycidyl methacrylate, silica gel, polyamide and polyester help improve the mechanical properties of the polymer carriers so that these can be used as carrier media for column chromatography.

Through the inductive heating of the column separating material the polymer changes its physical properties from originally very hydrophilic to relatively hydrophobic. This change has a significant effect on the separation and elution behavior of the carrier medium. As a result of the phase transition hydrophilic-hydrophobic, up to 60% more proteins such as albumin, fibronectin, fibrinogen and IgG-antibodies are normally retained on the separation column than before the phase transition. In addition, the separation characteristics of the separating medium can be significantly changed during a passage by switching the magnetic field on and consequently used to enable a better separation quality with substances that are otherwise difficult to separate. Examples here include the separation of proteins, oligonucleotides with only slightly different molecular masses as well as the separation of steroids whose retention times above the phase transition temperature generally increase by up to 70%.

Other magnetic core polymers that are suitable to produce thermosensitive polymer carriers are substrates that are biodegradable or have a high biocompatibility. This means that the in vivo application of the carrier matrix in particular can be significantly improved. Examples of such substrates are dextrane, gelatin, polylactides, polyglycolids, silica gels, starch, chitosan, albumin, polycyanacrylate, alginate, polyvinyl alcohol, agarose, polyethylene glycols and polyethylene oxides. The production of such magnetic basic polymers is explained in the aforementioned references.

The magnetic core polymers are introduced into the matrix in two different ways:
(a) through the radical or radiation-induced grafting of N-isopropylacrylamide; and b) through a simple polymerization of the core polymers during the synthesis.

The coating of polymer substrates by means of radiation-induced and radical grafting in the presence of cerium(IV) salts is generally known from the state of the art. It is normally carried out with aqueous 10 to 30% N-isopropyl-acrylamide solutions using a radiation dose of 0.2 to 1 Mrad (2 to 10 kGy) or in the presence of a 0.05 to 0.4 molar cerium(IV) saline solution. The corresponding methods can be found in: DE-OS 4129901, DE-OS 3811042, Müller-Schulte and Horster, Polymer Bull., Vol. 7, 77, 1982, Müller-Schulte and Thomas, Radiat. Phys. Chem., Vol. 35, 93, 1990, Müller-Schulte, Radiat. Phys. Chem., Vol. 42, 891, 1993, Tripathy et al., J. Appl. Polymer Sci., Vol. 81, 3296, 2001, Gupta et al., Biomacromolecules, Vol. 2, 239, 2001, Matsuoka et al., Polym. Gels & Networks, Vol. 6, 319, 1998, and Li et al., Radiat. Phys. Chem., Vol. 55, 173, 1999, so that they can be used by an expert in this field at any time.

Analogous to the production of the polymer carrier using inverse suspension polymerization, it was surprisingly discovered that during grafting with N-isopropylacrylamide, the co-grafting with monomers containing carboxyl groups such as acrylic acid or methacrylic acid is also particularly advantageous since the carboxyl groups that are introduced lead to drastically improved dynamics of shrinkage compared to the pure graft polymers. Thus, N-isopropylacrylamide grafted core polymers that cannot be swollen in water such as polyethylene, polypropylene, polyamide, polyester, polymethyl methacrylate, polyglycidyl methacrylate with a grafting degree of >40% and an acrylic acid share of 1-5% by mol normally have shrinkage values of 50% to 75% when heated from 30° to 45° C. whereas the shrinkage degrees with an acrylic acid content of <1% by mol are all below 50%. With otherwise constant N-isopropylacrylamide-acrylic acid mol ratios in the graft formulation the degrees of shrinkage increase with an increasing overall grafting degree.

The core polymers are produced by means of the known emulsion, suspension or precipitation polymerization or by suspension cross-linkage that are described in the following publications: Li et al., J. Microencapsulation, Vol. 15, 163, 1998, Quellec et al., J. Biomed. Mat. Res. Vol. 42, 45, 1998, Hua et al., J. Mater. Sci. Vol. 36, 731, 2001, Kriwet et al., J. Contr. Release, Vol. 56, 149, 1998, Chu et al., Polym. Bull., Vol. 44, 337, 2000, “Methods in Enzymology”, Vol. 112, Part A, Widder and Green editors, Academic Press, Inc., Orlando, 1985.

The grain sizes of the core polymers can be set to between 50 nm and 1000 nm depending on the requirements.

An essential feature of this present invention is the definition of the desired properties of the polymer carriers such as magnetic properties, functionality or porosity through the composition of the initial mixture. The porosity, an important influencing variable for the release behavior of the encapsulated active agents, is primarily determined by the concentration of the cross-linking agent in the monomer formulation. The monomer formulation normally contains between 0.1-10% cross-linking agent (relative to the total monomer content), preferably between 0.5% and 5%. Cross-linking agent concentrations of <1% are normally used to produce highly porous carriers (pore width >50 nm). Generally, bi- or tri-functional monomers that form a static copolymer with the monomer mixture can be used as cross-linking agents. Examples of such bi- and tri-functional monomers include N,N′-methylene bisacrylamide, ethylene glycol dimethacrylate, 1,1,1,-tris-(hydroxymethyl)propane triacrylate, 3-(acryloyloxy)-2-hydroxypropyl methacrylate, methacrylic acid allyl ester and acrylic acid vinyl ester.

The generally known radical agents are used to initiate the polymerization. Polymerization can be significantly accelerated through a combined addition of N,N,N′,N′-tetramethylethylene diamine (TEMED) and ammonium persulphate (APS). The concentrations of TEMED and APS (normally 10-40% aqueous solutions) relative to the monomer phase are between 2-8% by volume for TEMED and 2-10% by volume for APS, whereby an increasing concentration of TEMED and APS is normally accompanied by a proportionate rise in the speed of polymerization. In this way, the polymerization and thus the polymer particle formation can be completed within a matter of minutes, a process that normally takes up to 24 hours according to the state of the art.

For biomedical applications it has proven advantageous to copolymerize poly-N-isopropylacrylamide with those functional vinyl monomers that have a group suitable for coupling. Co-monomers that can be polymerized with N-isopropylacrylamide and have groups suitable for coupling in the form of amino, carboxyl, epoxy, hydroxyl, isothiocyanate, isocyanate or aldehyde functions are suitable here. Examples of these that in no way restrict the invention include: acrylic acid, methacrylic acid, acrylamide, 2-hydroxyethyl methacrylate, 2-isocyanatoethyl methacrylate, acrolein, hydroxypropyl methacrylate, 2-carboxyisopropyl acrylamide.

This type of copolymerisation opens up the possibility of coupling bioaffine ligands such as antibodies, cell receptors, anti-cell receptor antibodies, nucleic acid, oligosaccharides, lectins and antigens to the polymer carrier with which the thermosensitive carriers can be an directed to certain target substances such as cells, biomolecules, viruses, bacteria or tissue compartments and/or selectively attached to these target organs according to the principle of affinity. The polymer carriers can thus be attached specifically to T-cells, B-lymphocytes, monocytes, granulocytes, parent cells and leukocytes by coupling antibodies that are directed against the cell surface structures such as CD2, CD3, CD4, CD8, CD19, CD14, CD15, CD34 and CD45 (“cluster of differentiation”).

The specific application of the polymer carrier in accordance with the invention in conjunction with an externally controllable structural change surprisingly opens up the possibility of exploiting new integral active combinations. These consist of using the polymer particles as a new type of contrast-intensifying medium in the context of NMR diagnostics and parallel to this as a basis for a controllable application of active agents. From the state of the art, it is known (DE-OS 3508000, U.S. Pat. Nos. 5,492,814 and 4,647,447), that superparamagnetic, ferromagnetic or paramagnetic substances lead to a substantial intensification of the contrast during imaging in the context of NMR diagnostics (e.g. magnetic resonance tomography, MRT) which in turn enables a more precise diagnosis through a better localization and classification of pathological processes (e.g. detection of tumors in early stages and micro-metastases).

In conjunction with the coupling of bioaffine ligands to the polymer matrix, that allows a specific enrichment of the polymer particles in the (cell)tissue to be analyzed, the media in accordance with the invention can surprisingly be used almost in parallel as both carriers for therapeutic active agents as well as highly-sensitive diagnostic indicators.

Coupling those antibodies or antibody fragments that are oriented against a tumor cell antigen initially creates the precondition for selectively concentrating the polymer carrier in the tumor tissue and attaching this to the tumor cells. Examples of such tumor markers and/or antigens, though these do not restrict the invention, include: tumor-associated transplantation antigen (TATA), oncofetal antigen, tumor-specific transplantation antigen (TSTA), p53-protein, carcinoembryonic antigen (CEA), melanoma antigens (MAGE-1, MAGE-B2, DAM-6, DAM-10), mucin (MUC1), human epidermis receptor (HER-2), alpha-feto protein (AFP), helicose antigen (HAGE), human papilloma virus (HPV-E7), caspase-8 (CASP-8), CD3, CD10, CD20, CD28, CD30, CD25, CD64, interleukin-2, interleukin-9, mamma-CA antigen, prostate-specific antigen (PSA), GD2 antigen, melanocortin receptor (MCIR), 138H11 antigen. The corresponding antibodies can optionally be used as monoclonal or polyclonal antibodies, as antibody fragments (Fab, F(ab′)₂), as single-chain molecules (scFv), as “diabodies”, “triabodies”, “minibodies” or bispecific antibodies.

For the parallel treatment of tumors, the tumor agents and cytostatic agents known from cancer therapy are encapsulated in the polymer particles. Examples of these include: methotrexate, cis-platinum, cyclophosphamide, chlorambucil, busulphan, fluorouracil, doxorubicin, ftorafur or conjugates of these substances with proteins, peptides, antibodies or antibody fragments. Conjugates of this type are known from the state of the art: “Monoclonal Antibodies and Cancer Therapy, UCLA Symposia on Molecular and Cellular Biology, Reisfeld and Sell, Editors, Alan R. Riss, Inc., New York, 1985.

The known methods of coupling bioactive substances such as proteins, peptides, oligosaccharides or nucleic acids to solid carriers are used for the covalent binding of the bio- and affinity ligands or receptors to the polymer carrier (cf. “Methods in Enzymology”, Mosbach, editor, Vol 135, Part B, Academic Press, 1987). Coupling agents that are used here include, for example: tresyl chloride, tosyl chloride, cyanogen bromide, carbodiimide, epichlorhydrine, diisocyanate, diisothiocyanates, 2-fluoro-1-methyl-pyridinium-toluene-4-sulphonate, 1,4-butanediol-diglycidyl ether, N-hydroxysuccinimide, chlorine carbonate, isonitril, hydrazide, glutaraldehyde, 1,1′,-carbonyl-diimidazole. Moreover, the bioligands can also be coupled with reactive heterobifunctional compounds that can enter into a chemical bond with both the functional groups of the matrix (carboxyl, hydroxyl, sulfhydryl, amino groups) as well as the bioligands. Examples in the sense of the invention are: Succinimidyl-4-(N-maleiimido-methyl)-cyclohexane-1-carboxylate, 4-succinimidyloxycarbonyl-α-(2-pyridyldithio)toluene, succinimidyl-4-(p-maleimidophenyl)butyrate, N-γ-maleimidobutyryloxy succinimide, 3-(2-pyridyldithio)propionyl hydrazide, sulphosuccinimidyl-2-(p-azidosalicylamido)ethyl-1,3′-dithiopropionate. An expert in this field can use these coupling agents at any time in accordance with the information in “Ullmanns Encyclopädie der Technischen Chemie”, 4th Edition, Vol. 10, or G. T. Hermanson, “Bioconjugate Techniques”, Academic Press, San Diego, 1996.

The magnetic properties of the polymer particles are achieved by the direct admixture of a suitable magnetic colloid or metallic colloid or corresponding particles before dispersion into the monomer phase. Through a precise admixture of the colloids the heat-up behavior of the polymer particles can be selectively varied and/or adjusted. Thus, aqueous dispersions with a magnetic colloid share of 10% by weight can be heated up with a magnetic field amplitude of 30 kA/m and a frequency of 0.8 MHz within 30 seconds from room temperature to approximately 45° C. The heat-up values rise analogously with correspondingly higher magnetic colloid shares. These measurements relate to the heat-up rates recorded macroscopically in the dispersion. The actual heat generated in the polymer particles is consequently much higher. For the application of the media in accordance with the invention this means that only a very short induction period of a few seconds is adequate to create a stimulus triggered by heating. The poly-N-isopropylacrylamide gels already display a significant shrinkage at temperatures >27° C. that can be up to 85% relative to the original volume depending on the composition of the gel. The degree of shrinkage here depends on both the co-monomer content and type of co-monomers, as described above, as well as the degree of cross-linking. Thus, gels with a degree of cross-linking of <1 Mol % normally have a degree of shrinkage of 60% to 85% whereas that of gels with a degree of cross-linking of >1 Mol % is below 60%.

In order to heat the aforementioned magnetic and metallic substances and compounds up to the temperatures that are relevant for the analytical, therapeutic and diagnostic applications, a special design of the magnetic field is required with respect to the magnetic field strength and frequency. Current-carrying coils are normally used that are fed from a high-frequency generator. This type of coil system and high-frequency generator is state-of-the-art and are available commercially. The coil dimensions depend on the sizes of the respective samples; they are generally 5 to 30 cm in diameter and 5-30 cm long. The necessary output of the HF generators is normally between 0.5 and 1.5 kW. Two generator settings can in principle be used to heat up the magnetic samples: a) a high frequency in the range 5-20 MHz with a low magnetic field strength of 100-500 A/m or, b) a low frequency of 0.2-0.8 MHz in combination with a high field strength of 1 to 45 kA/m. Both field parameter combinations in principle guarantee a sufficient thermal output within a short application period (<1 min.). Sufficient energy to heat up the carrier can be also provided with larger coil geometries (30-40 cm diameter) by a corresponding increase in the field strength to >15 kA/m for the radiation of areas with a larger volume, as is the case for example in the application of medical active agents in certain parts of the body.

On account of the special combination of product and process, the polymer carriers in accordance with the invention can be used, in particular as a matrix, for the encapsulation of active agents and as media to block blood vessels.

Through the use of inductive heating, dosing systems for the administration and application of active agents can be created for the medical field or analytics that are characterized in particular by their contact-free controllability. An active agent is understood as meaning a substance that triggers a chemical, biochemical or physiological reaction in one way or another and hereby creates a therapeutic, diagnostic and/or prophylactic effect or can fulfill an analytical function. Examples include biologically active proteins or peptides, enzymes, antibodies, antigens, nucleic acids, glycoproteins, lectins, oligosaccharides, hormones, lipids, growth factors, interleukins, cytokines, steroids, vaccines, anticoagulants, cytostatic agents, immunomodulatory agents or antibiotics.

To this end, the active agents are encapsulated in the polymer particles. This is carried out either by a direct admixing of the corresponding active agent in the monomer mixture or through incubation of the active agent with the polymer carrier that has been shrunk beforehand through heat treatment. The concentration gradient towards a polymer gel produced by the shrinkage process causes the active agent to diffuse inside the gel.

The problem with the first encapsulation variant is that the partly very sensitive active agents such as proteins, antibodies or hormones are damaged or inactivated in some way by the polymerisation conditions. To combat this problem, it was surprisingly discovered that the addition of polyalcohols, sugars, serum albumin and gelatine is helpful since these can permanently stabilise the active agents against the effects of polymerisation. Examples of such substances, whose concentration in the monomer formulation is usually between 0.1 and 5% by weight, are: inosite, polyvinyl alcohol, mannite, sorbite, aldonite, erythrite, sucrose, glycerine, xylitol, fructose, glucose, galactose or maltose.

The carriers charged with the corresponding active agent that are produced in this way can then be applied to the desired physiological or bio-analytical sites of action with the aid of known administration methods such as injection, implantation, infiltration, diffusion, streaming or biopsy. The local application of the magnetic particles can be further intensified by positioning the particles exactly at the desired spots using electro- or strong permanent magnets that are placed over the reaction area or site of action from the outside. Once the polymer particles have reached their site of action they can be heated up to the corresponding phase transition temperature by applying a high-frequency magnetic alternating field that is located outside the actual site of action and/or reaction of the polymer carrier. The heat that is generated induces a shrinkage process in the polymer gel that triggers a rapid release of the encapsulated active agents from the matrix.

The times needed by the active agents to diffuse out of the gel in principle depend on the size of the gel, the molar weight of the active agent, the temperature of the gel and the degree of cross-linking of the carrier. It can generally be said that lower cross-linked gels (0.1 to 1% degree of cross-linking), as well as nano and microparticles, allow a faster diffusion of the active agent than higher cross-linked polymers (>1% degree of cross-linking) or macroscopic gels. Thus, 80% of low-molecular hormones such as vasopressin, insulin, testosterone, cortisone as well as antibiotics, cytostatic agents (molecular weight <10 kDa) diffuse out of a 1% cross-linked nanoparticle, mean particle size 430 nm, within one minute when heated to >40° C., whereas the same active agents in an approximately 5 μm gel particle require around 5 to 10 min. High-molecular active agents such as albumin, IgG-antibodies, fibrinogen, lactate dehydrogenase require correspondingly longer times under analogous conditions: >10 minutes. In order to alter the release rates of the active agents, the media in accordance with the invention as described above offer a number of adjustable and changeable parameters such as particle size, co-monomer content, type of co-monomers, heating and/or degree of cross-linking, that can alter the properties of the carrier medium to allow an optimum adjustment to the respective task.

In connection with the magnetic induction, this for the first time creates the basis for exploiting the change in structural properties of polymer carriers to allow a contact-free, controllable active agent application.

The media and processes in accordance with the invention also allow an inverse use of the swelling behaviour of the carrier by starting from a carrier that has been greatly shrunk in advance by heating that is then returned to its original swollen shape and/or geometry by a cooling process to below the phase transition temperature. This phenomenon can be applied in the context of therapeutic anti-tumor measures. One of the fatal pathological developments during tumor development is angiogenesis. This is generally understood as being a great dissemination in the formation of blood vessels in the tumor tissue. This pathological process, that up to now has been primarily treated with drugs (or by operations), can now be surprisingly suppressed or greatly delayed with the aid of the media in accordance with the invention. Particles, preferably with a particle size of 0.3 μm to 5 μm, that have been heated in advance per induction to temperatures >45° C. and have thus reached their maximum degree of shrinkage, are introduced into the tumor tissue. As a result of the subsequent adaptation to the body temperature the particles start to swell and reach their equilibrium swelling status after a few minutes. In this swollen state the polymer carriers have an embolising function, i.e. they are able to block the blood vessels and thus combat the development of tumors.

This special function is displayed in particular by those polymer particles whose phase transition temperature has been increased, for example by copolymerisation. Particularly suitable carriers are those which, as explained above, have co-monomers containing carboxyl groups. Carriers with a co-monomer content between 0.05 and 1% by mol and whose maximum shrinkage temperature is above 40° C. are given preference in this case. Particles with a particularly wide range of sizes are suitable to combat angiogenesis in practice since they allow blood vessels of all widths to be blocked at once.

The invention will be explained in more detail on the basis of the following examples.

### EXAMPLE 1

10 ml of a 0.1 M Na-phosphate buffer, pH 7.2, containing 15% N-isopropylacrylamide recrystallized from n-hexane, 5% acrylamide and 0.6% N,N′-methylene bisacrylamide, as well as 2.5 ml of an aqueous magnetic colloid containing 2.2 mM Fe/ml (mean particle size 26 nm) produced in accordance with a specification from Shinkai et al., Biocatalysis, Vol. 5, 61, 1991, are mixed and exposed to ultrasound for 5 min. in an ultrasonic bath (250 W) whilst being cooled with ice. Nitrogen is then introduced into the mixture for 15 min. to remove excess oxygen. 1 ml of an aqueous solution consisting of 0.1 mg anti-p53-antibodies (Roche Molecular Biochemicals), 0.05% Human Serum Albumin, 2% inosite and 0.5% gelatine is added to this mixture. It is exposed to ultrasound for a further 30 sec. whilst being cooled with ice. The aqueous phase is then mixed with 2 ml of a 30% ammonium persulphate solution (APS) containing 0.5% Igepal 720 in the presence of nitrogen and then suspended in 150 ml trichloroethylene that has been gassed for 20 min. beforehand with nitrogen and contains 1.5% of a mixture consisting of 80% Span 85 and 20% Tween 20, in a thermal controlled dispersing vessel (Ultra-Turrax LR 1000, IKA Werke) at 4° C. while being stirred (15,000 rpm). 1 ml of N,N,N′,N′-tetramethylethylenediamine (TEMED) is added after 10 sec. The suspension process is continued for 5 min. with a constant supply of nitrogen and ice cooling. The dispersion is left for a further 20 min. without stirring at 10° C. to polymerize. The dispersion is then placed in a glass column densely packed with steel wool (filling volume: approximately 10 ml; inside diameter: 0.5 cm) that is surrounded by a 5 cm long, ring-shaped neodymium-boron-iron-magnet and the mixture allowed to slowly (0.5 ml/min.) drip through the column. After this passage it is rinsed ten times with approximately 20 ml of Na-phosphate buffer containing 10% ethanol, 2% inosite and 1.5% polyvinyl alcohol (molecular weight, M_{w}: 5000). This is followed by washing five times in distilled water, and washing three times in 0.05 M Na-phosphate/1% inosite buffer, pH 7.2. The magnetic polymer fraction on the column is then eluted with 5 ml of a 0.1 M Na-phosphate buffer, pH 7.2, after removing the magnet. The eluate obtained in this way is then freeze dried. Following redispersion in 2 ml of a 0.05 Na-phosphate/0.1% Human Serum Albumin (HSA)/0.1% polyethylene glycol (PEG, M_{w}: 1000) buffer, pH 7.5, magnetic polymer particles with a mean particle size of 170 nm are obtained. The particles obtained are reduced in size by 43% within two minutes following treatment in a magnetic alternating field (magnetic field: 30 kA/m; 0.6 MHz, coil diameter: 5.5 cm, 8 windings).

The particles obtained in this way can be used as a contrast-intensifying medium in the context of NMR diagnostics and for the treatment of tumours.

### EXAMPLE 2

Cobalt-ferrite-nanoparticles (CoFe₂O₄) are produced according to a specification from Sato et al., J. Magn. Magn. Mat., Vol. 65, 252, 1987, from CoCl₂ and FeCl₃ and dispersed in water with the aid of a high-power ultrasonic finger (make: Dr. Hielscher, 80% amplitude) in the presence of 0.75% polyacrylic acid (M_{w}: 5.500) for 30 sec. 5 ml of the colloid containing 1.9 mM Fe/ml with a particle size of 21 nm are then mixed with 20 ml high-purity and degassed water in which 15% N-isopropylacrylamide, 6% acrylamide, 1% acrylic acid, 0.5% Igepal 520 and 0.8% N,N′-methylene bisacrylamide have been dissolved. The mixture is once again exposed to ultrasound for one min. with the ultrasonic finger whilst being cooled with ice and then in an ultrasonic bath for 30 min. After adding 2 ml of 40% APS, the mixture is dispersed in 300 ml of 1,1,1-trichloroethane containing 6% of a mixture of Tween 80 and Span 85 (72%:28%) with the aid of a dispersing machine (Ultra-Turrax, IKA Werke, 10,000 rpm) with ice cooling and the introduction of nitrogen. 1 ml of TEMED is added after 10 sec. The dispersion process is continued for 5 min. The reaction mixture is then left to complete the reaction for a further 20 min. at 10° C. The product is then separated and washed analogous to Example 1. After elution with 5 ml of 0.1 M Na-phosphate buffer, pH 7.4, it is dialyzed with 5 litres of a 0.01 M Na-phosphate buffer, pH 7.4, for 3 days. Magnetic particles with a mean particle size of 245 nm are obtained. 2 ml of the magnetic particle fraction obtained are placed in the magnetic separation column (cf. Example 1) and washed three times with a 0.01 M HCl solution and five times with high-purity water. After removing the magnet, 2 ml of 0.1 M 2-morpholino-ethanesulphonic acid (MES)/0.5% PEG (M_{w}: 1000)-buffer, pH 4.2, are added to elute the magnetic particles on the column. 0.5 ml of a 0.1 M MES-buffer, pH 4.2, in which 0.2 mM of N-cyclohexyl-N′-(2-morpholinoethyl)-carbodiimide-methyl-p-toluene sulphonate have been dissolved, are added to the eluate. The mixture is shaken lightly for 30 min. at room temperature. A subsequent passage through the separating column filled with steel wool separates off any excess N-cyclohexyl-N′-(2-morpholinoethyl)-carbodiimide-methyl-p-toluene sulphonate and the retained magnetic particle fraction is then washed five times with 15 ml of ice water in each case. After removing the magnet it is eluted with 1.5 ml of 0.05 M MES-buffer, pH 5.5. The eluate is mixed with 0.5 ml of the same MES-buffer in which the 1.25·10⁴ mM Anti-CD30-Fab-fragments are dissolved and coupled with the antibody fragments over a period of 12 hours at 4° C. The conjugate is separated over the column filled with steel wool and rinsed ten times with 10 ml of ice cold 0.05 M Na-phosphate/1% inosit/0.1% HSA-buffer, pH 7.2 in each case. This is followed by washing five times in 0.05 M glycine-buffer, pH 10.5 and washing two times in distilled water. The magnetic fraction is eluted with 2 ml of a 0.1 M Tris/HCl buffer, pH 8.5, after removing the hand magnet. The eluate is incubated with 3 ml of Tris buffer containing 1 M glycine, pH 8.5, for 12 hours at room temperature to deactivate any remaining carbodiimide. The magnetic fraction is then separated over the magnetic column and rinsed ten times with 0.05 M phosphate buffer/0.05% HSA, pH 7.5. After successful elution of the magnetic conjugate with 2 ml of 0.05 M phosphate buffer/0.05% HSA, pH 7.5, the magnetic particles can be used in accordance with the known application methods as contrast-intensifying media in the context of NMR diagnostics to diagnose Hodgkin's lymphoma.

### EXAMPLE 3

7.5 ml of a 0.1 M Na-phosphate buffer, pH 7.2, in which 20% N-isopropylacrylamide, 4% acrylamide, 1% N,N′-methylenebisacrylamide and 2.4% 2-hydroxyethyl-methacrylate have been dissolved, are rinsed for 20 min. with nitrogen and then mixed with 2.5 ml of a magnetite-ferrofluid (EMG 507, FerroTec, USA). The mixture is exposed to ultrasound in an ultrasonic bath for 5 min. whilst being cooled with ice. 2 ml of 1% gelatine and an insulin solution containing 0.1% HSA (INSUMANO Basal, 100 IU/ml) are then added. After adding 1.2 ml of a 35% APS solution to the aqueous phase this is dispersed in 130 ml of trichloroethylene containing 2.5% Span 60 and 1% Tween 80, with stirring (1200 rpm) and constant ice cooling as well as a continuous flow of nitrogen. After 20 sec., 0.5 ml of TEMED are added and the mixture stirred for 8 min. at 10° C. The reaction mixture is then left to complete the reaction for a further 20 min. at 15° C. The magnetic phase is separated and the retained product purified analogous to Example 1. After freeze drying and repeated dispersion in 2 ml of a Na-phosphate buffer/0.1% HSA/0.5% PEG (M_{w}: 1000), pH 7.2, magnetic particles with a mean particle size of 23 μm are obtained. Exposure of the particles to a magnetic field (15 kA/m; 0.6 MHz, coil diameter: 5.5 cm, 8 windings) leads to a 55% shrinkage within 3 min., whereby 58% of the originally added insulin is released. The polymer carriers can be used as an insulin depot in the treatment of diabetes.

### EXAMPLE 4

1 g of polyvinyl alcohol particles containing 40% by weight of magnetite (mean particle size 25 μm), that have been produced in accordance with a specification from Müller-Schulte and Brunner, J. Chromatogr. A 711, Vol. 711, 53, 1995, are mixed with 4 ml of a 20% aqueous N-isopropylacrylamide solution, 0.5 ml N-vinylpyrrolidone, 3 ml acetone and 5 ml methanol. The mixture is then rinsed for 20 minutes with argon, followed by a 45 hour irradiation with gamma rays from a Cs137 source (Gammacell 40) (total dose 3.4 kGy). The grafted material is then extracted for 20 hours with ethanol, followed by a ten hour extraction with water. After drying to a constant weight, this produces a graft yield of 67% by weight (relative to the original polymer). Inductive heating to 40° C. leads to a degree of shrinkage of 62%. The carrier obtained in this way can be used in column chromatography to separate proteins

What has been described above are preferred aspects of the present invention. It is of course not possible to describe every conceivable combination of components or methodologies for purposes of describing the present invention, but one of ordinary skill in the art will recognize that many further combinations and permutations of the present invention are possible. Accordingly, the present invention is intended to embrace all such alterations, combinations, modifications, and variations that fall within the spirit and scope of the appended claims.

1. Thermosensitive polymers containing at least one of magnetic or metallic colloids, wherein said polymers are produced by inverse suspension polymerization and have a physical structure changeable by magnetic induction.

2. The thermosensitive polymers according to claim 1, wherein the polymers comprise at least one compound selected from the group consisting of poly-N-isopropylacrylamide, poly-N-substituted acrylamides, poly-N-substituted methacrylamides, and copolymers of monomers from the group consisting of N-isopropylacrylamide, N-substituted acrylamides and N-substituted methacrylamides.

3. The thermosensitive polymers according to claim 2, wherein the polymers contain at least one copolymer or block copolymer which contain at least one comonomer selected from the group of monomers containing carboxyl groups consisting of acrylic acid and methacrylic acid, or from the group consisting of acrylates, acrylate derivatives, methacrylates, methacrylate derivatives, acrolein, acrylamide, N-substituted acrylamides and vinyl acetate.

4. The thermosensitive polymers according to claim 2, wherein the polymers contain at least one copolymer or block copolymer selected from the group consisting of polyacrylic acid, polyacrolein, polymethacrylic acid, polyacrylamide and N-substituted polyacrylamides.

5. The thermosensitive polymers according to claim 1, wherein the polymers are selected from the group consisting of nano-particles and microparticles.

6. The thermosensitive polymers according to claim 1, wherein said magnetic induction is a high-frequency, magnetic alternating field.

7. The thermosensitive polymers according to claim 1, wherein the change in the physical structure is a change to the geometric form of the polymers.

8. The thermosensitive polymers according to claim 7, wherein the change in the geometric form is a return to the original form displayed by the polymers before a change in form caused by heat.

9. The thermosensitive polymers with according to claim 1, wherein the change in the physical structure is an enlargement or reduction in the size of the polymer particles.

10. The thermosensitive polymers according to claim 1, wherein the magnetic colloids comprise a material selected from the group consisting of ferromagnetic particles, superparamagnetic particles, ferrimagnetic particles, low-temperature-ferrites and a ferrofluid with a particle size of <1 μm.

11. The thermosensitive polymers according to claim 10, wherein the low-temperature-ferrites have a Curie temperature in the range of 30° C. to 100° C.

12. The thermosensitive polymers according to claim 1, wherein the metallic colloids comprise an element selected from the group consisting of 8, 9, 10 and 11 (group classification: new suggestion of the 1986 IUPAC definition.

13. The thermosensitive polymers according to claim 1, wherein a core polymer encapsulates the magnetic and/or or metallic colloids.

14. The thermosensitive polymers according to claim 13, wherein the core polymer has a particle size of 50 to 1000 nm.

15. The thermosensitive polymers according to claim 14, wherein said magnetic or metallic colloids encapsulated in the core polymer are in a disperse colloid form.

16. The thermosensitive polymers according to claim 13, wherein the encapsulating core polymer is selected from the group consisting of chitosan, dextran, starch, polyacrylic acid, polysaccharides, silica gel, silicone derivatives, cellulose, proteins, albumin, polyacrylic acids, agarose, alginate, polystyrene, polyacrylates, polymethacrylates, polycyanoacrylates, polymethyl methacrylate, polyvinyl alcohol, polyamides, polyesters, polyamino acids, hyaluronic acid, polylactides, polyglycolides, polyacrolein and copolymers of the same.

17. The thermosensitive polymers according to claim 1, wherein the polymers contain a porogen in an amount of 0.1-30% by weight.

18. The thermosensitive polymers according to claim 17, wherein the porogen is selected from the group consisting of silica gels, proteins, nucleic acids, polyethylene glycols, polyethylene oxides and polysaccharides.

19. The thermosensitive polymers according to claim 1, wherein the polymers are cross-linked with a bi- or tri-functional cross-linking agent.

20. The thermosensitive polymers according to claim 19, wherein the cross-linking agent has a concentration of 0.1% to 10% relative to the overall monomer content.

21. The thermosensitive polymers according to claim 1, wherein the polymers further contain reactive groups that bond biomolecules.

22. The thermosensitive polymers according to claim 21, wherein the bonding groups are reacted with a compound selected from the group consisting of affinity ligands, peptides, proteins, antibodies, antigens, enzymes, cell receptor antibodies, antibodies against tumor markers, antibody fragments, artificially produced antibodies, modified antibodies, antibody conjugates, oligosaccharides, glycoproteins, lectins, nucleic acids, streptavidin and biotin.

23. The thermosensitive polymers according to claim 1 wherein the polymers further contain at least one encapsulated active agent releasable from the polymer into the environment by exposure to a magnetic field.

24. The thermosensitive polymers according to claim 23, wherein the at least one encapsulated active agent is selected from the group consisting of hormones, cytostatic agents, antibodies, antibody derivatives, antibody fragments, cytokines, immunomodulators, antigens, proteins, peptides, lectins, glycoproteins, nucleic acids, antisense-nucleic acids, oligosaccharides, antibiotics and generic agents.

25. A process for the production of thermosensitive polymers in accordance with claim 1, comprising the steps of dispersing at least one of magnetic or metallic colloids in an aqueous monomer solution,
suspending said aqueous monomer solution through mechanical comminution in an organic phase that is not miscible with water after adding a multifunctional cross-linking agent and a radical initiator and;
radically polymerizing said organic phase to nano- or microparticles.

26. A process for the production of thermosensitive polymers in accordance with claim 1, comprising the steps of dispersing at least one of magnetic or metallic colloids in an aqueous monomer solution;
suspending said aqueous monomer solution is through mechanical comminution in an organic phase that is not miscible with water after adding a multifunctional cross-linking agent; and
adding a radical initiator to radically polymerize said organic phase to nano or microparticles during the suspension process.

27. The process for the production of thermosensitive polymers with according to claim 25, wherein said aqueous monomer solution comprises at least one monomer selected from the group consisting of N-isopropylacrylamide, N-substituted acrylamides, and N-substituted methacrylamides.

28. The process for the production of thermosensitive polymers in according to claim 25, and further comprising the step of adding 0.05 to 30% by mol co-monomers to the monomer solution.

29. The process for the production of thermosensitive polymers with according to claim 28, wherein the co-monomers are at least one compound selected from the group consisting of acrylate derivatives, methacrylate derivatives, acrylic acid, acrolein, methacrylic acid, acrylamide, and vinyl acetate.

30. The process for the production of thermosensitive polymers according to claim 25, and further comprising the step of adding a material selected from the group consisting of ferromagnetic, superparamagnetic or ferrimagnetic substances, low-temperature ferrites and ferrofluids with a particle size of <1 μm to the monomer solution.

31. The process for the production of thermosensitive polymers according to claim 30, wherein the ferromagnetic, superparamagnetic or ferrimagnetic substances or low-temperature ferrites are present as colloids or in a powder form.

32. Process The process for the production of thermosensitive polymers according to claim 25, and further comprising the steps of:
dispersively encapsulating said at least one magnetic or metallic colloids in a nano or microparticle core polyer; and
adding said encapsulation to the monomer solution.

33. The process for the production of thermosensitive polymers according to claim 32, wherein the core polymer comprises a compound selected from the group consisting of by chitosan, dextran, starch, polyacrylic acid, polysaccharides, silica gel, silicone derivatives, cellulose, proteins, albumin, polyacrylic acid, agarose, alginate, polystyrene, polyacrylates, polymethacrylates, polycyanoacrylates, polymethyl methacrylate, polyvinyl alcohol, polyamino acids, hyaluronic acid, polylactides, polyglycolides, polyacrolein and copolymers of the same.

34. The process for the production of thermosensitive polymers according to claim 25, wherein solvents are used as the organic phase and have a polar solubility parameter of 5-10 (cal/cm³)^{1/2}.

35. The process for the production of thermosensitive polymers according to claim 25, and further comprising the step of adding at least one surfactive substance to the organic phase at 0.05 to 15% by weight.

36. The process for the production of thermosensitive polymers according to claim 35, wherein the surface active substance is at least one compound selected from the group consisting of alkyl sulphosuccinates, polyoxyethylene aryl ethers, polyoxyethylenes, polyoxyethylene sorbitan esters, polyoxyethylene adducts, polyethylene propylene oxide block copolymers, alkylphenoxy polyethoxy ethanols, fatty alcohol polyethylene glycol ethers, polyglycerol esters, polyoxyethylene alcohols, polyoxyethylene sorbitan fatty acid esters, and polyoxyethylene acids.

37. The process for the production of thermosensitive polymers according to claim 25, and further comprising the step of pre-polymerizing the monomer solution for 5-120 seconds before dispersion in the organic phase.

38. The process for the production of thermosensitive polymers according to claim 25, and further comprising the step of bonding a compound selected from the group consisting of affinity ligands, peptides, proteins, antibodies, antigens, enzymes, cell receptor antibodies, antibodies against tumor markers, antibodies against tumor antigens, antibody fragments, artificially produced antibodies, modified antibodies, antibody conjugates, oligosaccharides, glycoproteins, lectins, nucleic acid, streptavidin and biotin are bonded to the polymers.

39. The process for the production of thermosensitive polymers according to claim 25, and further comprising the step of encapsulating the active agents in the polymers by adding the active agent(s) to a monomer solution containing at least one of magnetic or metallic colloids.

40. The process for the production of thermosensitive polymers according to claim 39, wherein the active agents are selected from the group consisting of hormones, cytostatic agents, antibodies, cytokines, immunomodulators, antigens, proteins, peptides, lectins, glycoproteins, nucleic acids, antisense-nucleic acids, oligosaccharides, antibiotics and generic agents.

41. The process for the production of thermosensitive polymers according to claim 40, and further comprising the step of adding a compound selected from the group consisting of polyvalent alcohols, polyvinyl alcohols, gelatins and carbohydrates are added to the active agents in an amount of 0.1 to 20% by weight.

42. The process for the production of thermosensitive polymers according to claim 41, wherein the polyvalent alcohols or carbohydrates are selected from the group consisting of inosite, mannite, sorbite, aldonite, erythrite, sucrose, glycerine, xylite, fructose, glucose, galactose and maltose.

43. A process for the release of active agents from active agent-containing particles, wherein the particles of thermosensitive polymers according to claim 1 or particles which have been produced according to a process of claim 25 comprising the step of introducing said particles into a magnetic alternating field magnetic induction.

44. A process for changing the physical structure of thermosensitive polymers containing at least one of magnetic or metallic colloids, or for warming or heating said polymers, comprising the step of introducing said polymers into a magnetic alternating field for magnetic induction.

45. The use of thermosensitive polymers containing at least one of magnetic or metallic colloids according to claim 1 or of particles produced by a process according to claim 25 as a contrast-intensifying media in NMR diagnostics, as carriers for active agents in medical therapy and diagnostics, as controllable carriers for reactants, as media to control microfluid processes, as separation media in column chromatography, as media to adjust and regulate pore sizes in membranes, as media to block blood vessels, as artificial cell carriers, as separation media for nucleic acids, cells, proteins, steroids, viruses or bacteria, in each case by using a magnetic alternating field, preferably a high-frequency magnetic alternating field.

46. The process according to claim 43, wherein said magnetic alternating field is a high-frequency magnetic alternating field.

47. The process according to claim 44, wherein said magnetic alternating field is a high-frequency magnetic alternating field.

48. The process for the production of thermosensitive polymers according to claim 26, wherein said aqueous monomer solution comprises at least one monomer selected from the group consisting of N-isopropylacrylamide, N-substituted acrylamides, and N-substituted methacrylamides.

49. The process for the production of thermosensitive polymers according to claim 26, and further comprising the step of adding 0.05 to 30% by mol co-monomers to the monomer solution.

50. The process for the production of thermosensitive polymers according to claim 49, wherein the co-monomers are at least one compound selected from the group consisting of acrylate derivatives, methacrylate derivatives, acrylic acid, acrolein, methacrylic acid, acrylamide, and vinyl acetate.

51. The process for the production of thermosensitive polymers according to claim 26, and further comprising the step of adding a material selected from the group consisting of ferromagnetic, superparamagnetic or ferrimagnetic substances, low-temperature ferrites and ferrofluids with a particle size of <1 μm to the monomer solution.

52. The process for the production of thermosensitive polymers according to claim 51, wherein the ferromagnetic, superparamagnetic or ferrimagnetic substances or low-temperature ferrites are present as colloids or in a powder form.

53. The process for the production of thermosensitive polymers according to claim 26, and further comprising the steps of:
dispersively encapsulating said at least one magnetic or metallic colloids in a nano or microparticle core polyer; and
adding said encapsulation to the monomer solution.

54. The process for the production of thermosensitive polymers according to claim 53, wherein the core polymer comprises a compound selected from the group consisting of chitosan, dextran, starch, polyacrylic acid, polysaccharides, silica gel, silicone derivatives, cellulose, proteins, albumin, polyacrylic acid, agarose, alginate, polystyrene, polyacrylates, polymethacrylates, polycyanoacrylates, polymethyl methacrylate, polyvinyl alcohol, polyamino acids, hyaluronic acid, polylactides, polyglycolides, polyacrolein and copolymers of the same.

55. The process for the production of thermosensitive polymers according to claim 26, wherein solvents are used as the organic phase and have a polar solubility parameter of 5-10 (cal/cm³)^{1/2}.

56. The process for the production of thermosensitive polymers according to claim 26, and further comprising the step of adding at least one surfactive substance to the organic phase at 0.05 to 15% by weight.

57. The process for the production of thermosensitive polymers according to claim 56, wherein the surface active substance is at least one compound selected from the group consisting of alkyl sulphosuccinates, polyoxyethylene aryl ethers, polyoxyethylenes, polyoxyethylene sorbitan esters, polyoxyethylene adducts, polyethylene propylene oxide block copolymers, alkylphenoxy polyethoxy ethanols, fatty alcohol polyethylene glycol ethers, polyglycerol esters, polyoxyethylene alcohols, polyoxyethylene sorbitan fatty acid esters, and polyoxyethylene acids.

58. The process for the production of thermosensitive polymers according to claim 26, and further comprising the step of bonding a compound selected from the group consisting of affinity ligands, peptides, proteins, antibodies, antigens, enzymes, cell receptor antibodies, antibodies against tumor markers, antibodies against tumor antigens, antibody fragments, artificially produced antibodies, modified antibodies, antibody conjugates, oligosaccharides, glycoproteins, lectins, nucleic acid, streptavidin and biotin to the polymers.

59. The process for the production of thermosensitive polymers according to claim 26, and further comprising the step of encapsulating the active agents in the polymers by adding the active agent(s) to a monomer solution containing at least one of magnetic or metallic colloids.

60. The process for the production of thermosensitive polymers according to claim 59, wherein the active agents are selected from the group consisting of hormones, cytostatic agents, antibodies, cytokines, immunomodulators, antigens, proteins, peptides, lectins, glycoproteins, nucleic acids, antisense-nucleic acids, oligosaccharides, antibiotics and generic agents.

61. The process for the production of thermosensitive polymers according to claim 60, and further comprising the step of adding a compound selected from the group consisting of polyvalent alcohols, polyvinyl alcohols, gelatins and carbohydrates to the active agents in an amount of 0.1 to 20% by weight.

62. The process for the production of thermosensitive polymers according to claim 61, wherein the polyvalent alcohols or carbohydrates are selected from the group consisting of inosite, mannite, sorbite, aldonite, erythrite, sucrose, glycerine, xylite, fructose, glucose, galactose and maltose.

63. A process for the release of active agents from active agent-containing particles, wherein the particles of thermosensitive polymers according to claim 1 or particles which have been produced according to a process of claim 26 comprising the step of introducing said particles into a magnetic alternating field for magnetic induction.

64. The process according to claim 63, wherein said magnetic alternating field is a high-frequency magnetic alternating field.

65. The use of thermosensitive polymers containing at least one of magnetic or metallic colloids according to claim 1 or of particles produced by a process according to claim 26 as a contrast-intensifying media in NMR diagnostics, as carriers for active agents in medical therapy and diagnostics, as controllable carriers for reactants, as media to control microfluid processes, as separation media in column chromatography, as media to adjust and regulate pore sizes in membranes, as media to block blood vessels, as artificial cell carriers, as separation media for nucleic acids, cells, proteins, steroids, viruses or bacteria, in each case by using a magnetic alternating field, preferably a high-frequency magnetic alternating field.
